(19) United States
(12) Patent Application Publication
(10) Pub. No.: US 2025/0025109 A1
(43) Pub. Date: Jan. 23, 2025

(54) MONITORING SYSTEM, WEARABLE TERMINAL, MONITORING METHOD, AND PROGRAM

(71) Applicant: TEIKYO UNIVERSITY, Tokyo (JP)

(72) Inventor: Jun KANDA, Tokyo (JP)

(21) Appl. No.: 18/716,796

(22) PCT Filed: Nov. 24, 2022
(86) PCT No.: PCT/JP2022/043376

§ 371 Date: Jun. 5, 2024

(30) Foreign Application Priority Data
Dec. 8, 2021 (JP) 2021-199338

(51) Int. Cl. A61B 5/00; A61B 5/0205; A61B 5/021; A61B 5/024; A61B 5/145; G16H 40/67

(52) U.S. Cl. CPC A61B 5/7282; A61B 5/0002; A61B 5/02055; A61B 5/746; G16H 40/67; A61B 5/021; A61B 5/02438; A61B 5/14542

(57) ABSTRACT

A monitoring system includes a wearable terminal and a management server, in which the wearable terminal includes a detection unit, a score calculation unit, an alert output unit, and a transmission unit, the management server includes a reception unit and an output unit, in a case where a severity evaluation score calculated based on biological information of a wearer of the wearable terminal is equal to or more than a threshold value, the alert output unit outputs a consciousness disorder evaluation alert, the detection unit detects the biological information of the wearer of the wearable terminal and a reaction of the wearer of the wearable terminal to the output consciousness disorder evaluation alert, and the score calculation unit calculates the severity evaluation score including an evaluation of a consciousness disorder based on the detected biological information of the wearer of the wearable terminal and the detected reaction of the wearer of the wearable terminal to the consciousness disorder evaluation alert.

### TECHNICAL FIELD

The present invention relates to a monitoring system, a wearable terminal, a monitoring method, and a program.

Priority is claimed on Japanese Patent Application No. 2021-199338, filed on Dec. 8, 2021, the content of which is incorporated herein by reference.

### BACKGROUND ART

Patent Document 1 describes a watching system including a wearable terminal and a management server. In the technology disclosed in Patent Document 1, the wearable terminal is worn by a target person to be watched, such as an elderly person, and a biological sensor of the wearable terminal measures biological data of the person to be watched, such as a heartbeat pattern and a heartrate.

Meanwhile, in the technology disclosed in Patent Document 1, the biological data of the target person to be watched (a wearer of the wearable terminal) is measured, and monitoring is not performed in consideration of the presence or absence of a consciousness disorder of the wearer of the wearable terminal. Therefore, with the technology disclosed in Patent Document 1, there is a concern that the wearer of the wearable terminal cannot be appropriately monitored.

On the other hand, if an alert for a consciousness disorder evaluation used for evaluating the consciousness disorder of the wearer of the wearable terminal is output excessively, there is a risk in which the wearer of the wearable terminal feels the alert for the consciousness disorder evaluation to be annoying.

In addition, if the evaluation of the consciousness disorder of the wearer of the wearable terminal is performed excessively, there is also a risk in which evaluation accuracy of the consciousness disorder of the wearer of the wearable terminal is reduced.

### CITATION LIST

### Patent Document

### [Patent Document 1]

Japanese Unexamined Patent Application, First Publication No. 2017-091076

### SUMMARY OF INVENTION

### Technical Problem

In view of the problems described above, an object of the present invention is to provide a monitoring system, a wearable terminal, a monitoring method, and a program capable of appropriately monitoring a wearer of a wearable terminal in a necessary case based on a severity evaluation score including an evaluation of a consciousness disorder while reducing an output of an alert for a consciousness disorder evaluation at a necessary minimum.

### Solution to Problem

According to one aspect of the present invention, there is provided a monitoring system that includes a wearable terminal and a management server to monitor a wearer of the wearable terminal, in which the wearable terminal includes: a detection unit configured to detect biological information of the wearer of the wearable terminal; a score calculation unit configured to calculate a severity evaluation score used for evaluating a severity of the wearer of the wearable terminal, based on the biological information of the wearer of the wearable terminal detected by the detection unit; an alert output unit configured to output a consciousness disorder evaluation alert used for evaluating a consciousness disorder of the wearer of the wearable terminal; and a transmission unit configured to transmit the severity evaluation score calculated by the score calculation unit, and the biological information of the wearer of the wearable terminal used for calculating the severity evaluation score, to the management server, the management server includes: a reception unit configured to receive the severity evaluation score, and the biological information of the wearer of the wearable terminal used for calculating the severity evaluation score, which are transmitted by the transmission unit; and an output unit configured to output the severity evaluation score, and the biological information of the wearer of the wearable terminal used for calculating the severity evaluation score, which are received by the reception unit, and in a case where the severity evaluation score, which does not include the evaluation of the consciousness disorder, calculated by the score calculation unit is equal to or more than a threshold value, the alert output unit outputs the consciousness disorder evaluation alert, the detection unit detects the biological information of the wearer of the wearable terminal and a reaction of the wearer of the wearable terminal to the consciousness disorder evaluation alert output by the alert output unit, the score calculation unit calculates the severity evaluation score which includes the evaluation of the consciousness disorder, based on the biological information of the wearer of the wearable terminal and the reaction of the wearer of the wearable terminal to the consciousness disorder evaluation alert output by the alert output unit, which are detected by the detection unit, and the transmission unit transmits the severity evaluation score, which includes the evaluation of the consciousness disorder, calculated by the score calculation unit, and the biological information of the wearer of the wearable terminal and the reaction of the wearer of the wearable terminal to the consciousness disorder evaluation alert used for calculating the severity evaluation score which includes the evaluation of the consciousness disorder, to the management server.

According to another aspect of the present invention, there is provided a wearable terminal used for monitoring a wearer of the wearable terminal, the wearable terminal including: a detection unit configured to detect biological information of the wearer of the wearable terminal; a score calculation unit configured to calculate a severity evaluation score used for evaluating a severity of the wearer of the wearable terminal, based on the biological information of the wearer of the wearable terminal detected by the detection unit; an alert output unit configured to output a consciousness disorder evaluation alert used for evaluating a consciousness disorder of the wearer of the wearable terminal; and a transmission unit configured to transmit the severity evaluation score calculated by the score calculation unit, and the biological information of the wearer of the wearable terminal used for calculating the severity evaluation score, to a management server, in which in a case where the severity evaluation score, which does not include the evaluation of the consciousness disorder, calculated by the score calculation unit is equal to or more than a threshold value, the alert output unit outputs the consciousness disorder evaluation alert, the detection unit detects the biological information of the wearer of the wearable terminal and a reaction of the wearer of the wearable terminal to the consciousness disorder evaluation alert output by the alert output unit, the score calculation unit calculates the severity evaluation score which includes the evaluation of the consciousness disorder, based on the biological information of the wearer of the wearable terminal and the reaction of the wearer of the wearable terminal to the consciousness disorder evaluation alert output by the alert output unit, which are detected by the detection unit, the transmission unit transmits the severity evaluation score, which includes the evaluation of the consciousness disorder, calculated by the score calculation unit, and the biological information of the wearer of the wearable terminal and the reaction of the wearer of the wearable terminal to the consciousness disorder evaluation alert used for calculating the severity evaluation score which includes the evaluation of the consciousness disorder, to the management server, a reception unit of the management server receives the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are transmitted by the transmission unit, and an output unit of the management server outputs the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are received by the reception unit.

According to still another aspect of the present invention, there is provided a monitoring method of monitoring a wearer of a wearable terminal, the monitoring method including: a detection step of detecting biological information of the wearer of the wearable terminal, via the wearable terminal; a score calculation step of calculating a severity evaluation score used for evaluating a severity of the wearer of the wearable terminal, based on the biological information of the wearer of the wearable terminal detected in the detection step, by the wearable terminal; an alert output step of outputting a consciousness disorder evaluation alert used for evaluating a consciousness disorder of the wearer of the wearable terminal, by the wearable terminal; a transmission step of transmitting the severity evaluation score calculated in the score calculation step, and the biological information of the wearer of the wearable terminal used for calculating the severity evaluation score, to a management server, by the wearable terminal; a reception step of receiving the severity evaluation score, and the biological information of the wearer of the wearable terminal used for calculating the severity evaluation score, which are transmitted in the transmission step, by the management server; and an output step of outputting the severity evaluation score, and the biological information of the wearer of the wearable terminal used for calculating the severity evaluation score, which are received in the reception step, by the management server, in which in a case where the severity evaluation score, which does not include the evaluation of the consciousness disorder, calculated in the score calculation step is equal to or more than a threshold value, in the alert output step, the consciousness disorder evaluation alert is output, in the detection step, the biological information of the wearer of the wearable terminal and a reaction of the wearer of the wearable terminal to the consciousness disorder evaluation alert output in the alert output step are detected, in the score calculation step, the severity evaluation score which includes the evaluation of the consciousness disorder is calculated, based on the biological information of the wearer of the wearable terminal and the reaction of the wearer of the wearable terminal to the consciousness disorder evaluation alert output in the alert output step, which are detected in the detection step, and in the transmission step, the severity evaluation score, which includes the evaluation of the consciousness disorder, calculated in the score calculation step, and the biological information of the wearer of the wearable terminal and the reaction of the wearer of the wearable terminal to the consciousness disorder evaluation alert used for calculating the severity evaluation score which includes the evaluation of the consciousness disorder are transmitted to the management server.

According to still another aspect of the present invention, there is provided a program causing a computer mounted in a wearable terminal used for monitoring a wearer of the wearable terminal to execute: a detection step of detecting biological information of the wearer of the wearable terminal; a score calculation step of calculating a severity evaluation score used for evaluating a severity of the wearer of the wearable terminal, based on the biological information of the wearer of the wearable terminal detected in the detection step; an alert output step of outputting a consciousness disorder evaluation alert used for evaluating a consciousness disorder of the wearer of the wearable terminal; and a transmission step of transmitting the severity evaluation score calculated in the score calculation step, and the biological information of the wearer of the wearable terminal used for calculating the severity evaluation score, to a management server, in which in a case where the severity evaluation score, which does not include the evaluation of the consciousness disorder, calculated in the score calculation step is equal to or more than a threshold value, in the alert output step, the consciousness disorder evaluation alert is output, in the detection step, the biological information of the wearer of the wearable terminal and a reaction of the wearer of the wearable terminal to the consciousness disorder evaluation alert output in the alert output step are detected, in the score calculation step, the severity evaluation score which includes the evaluation of the consciousness disorder is calculated, based on the biological information of the wearer of the wearable terminal and the reaction of the wearer of the wearable terminal to the consciousness disorder evaluation alert output in the alert output step, which are detected in the detection step, in the transmission step, the severity evaluation score, which includes the evaluation of the consciousness disorder, calculated in the score calculation step, and the biological information of the wearer of the wearable terminal and the reaction of the wearer of the wearable terminal to the consciousness disorder evaluation alert used for calculating the severity evaluation score which includes the evaluation of the consciousness disorder are transmitted to the management server, a reception unit of the management server receives the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are transmitted in the transmission step, and an output unit of the management server outputs the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are received by the reception unit.

### Advantageous Effects of Invention

According to the present invention, it is possible to provide a monitoring system, a wearable terminal, a monitoring method, and a program capable of appropriately monitoring a wearer of a wearable terminal in a necessary case based on a severity evaluation score including an evaluation of a consciousness disorder while reducing an output of an alert for a consciousness disorder evaluation at a necessary minimum.

### BRIEF DESCRIPTION OF DRAWINGS

FIG. 1 is a diagram showing an example of a monitoring system 1 according to a first embodiment.

FIG. 2 is a diagram showing an example of a severity evaluation score calculated by a score calculation unit 1A3.

FIG. 3 is a sequence diagram showing an example of a process executed in the monitoring system 1 according to the first embodiment.

FIG. 4 is a diagram showing a relationship between the severity evaluation score, the number of patients, an occurrence rate of Heat stroke (core body temperature of 40° C. or higher and GCS of 8 or lower), and the number of cases in an example of the monitoring system 1 according to the first embodiment.

FIG. 5 is a diagram comparing relationship between the severity evaluation score (horizontal axis) and the occurrence rate of Heat stroke (core body temperature of 40° C. or higher and GCS of 8 or lower) (vertical axis) in the example of the monitoring system 1 according to the first embodiment with a score-before-alert and a score-after-alert.

FIG. 6 is a diagram showing a relationship between the severity evaluation score, the number of patients, a severe case rate of a disease, and the number of cases in the example of the monitoring system 1 according to the second embodiment.

FIG. 7 is a diagram comparing a relationship between the severity evaluation score (horizontal axis) and the severe case rate of the disease (vertical axis) in the example of the monitoring system 1 according to a second embodiment with the score-before-alert and the score-after-alert.

FIG. 8 is a diagram showing a disease to which the monitoring system 1 of the second embodiment is applied for monitoring a severity in the example of the second embodiment of the monitoring system 1.

### DESCRIPTION OF EMBODIMENTS

Before showing embodiments of a monitoring system, a wearable terminal, a monitoring method, and a program according to the present invention, a background or the like in which the wearable terminal is used will be described.

The wearable terminal is a type of terminal that is worn on a body. The wearable terminal is generally, for example, a wristwatch-type wearable terminal, a glasses-type wearable terminal, a camera-type wearable terminal, or the like. The wristwatch-type wearable terminal includes a “watch type” and a “wristband type”. The “watch type” wristwatch-type wearable terminal is a type of terminal called a “smart watch”, and can check incoming of a mail, SNS, and the like without taking out a smartphone main body by cooperating with the smartphone. The “wristband type” wristwatch-type wearable terminal is a wearable terminal having a shape of a wristband, and is mainly used as an activity meter for the purpose of recording daily life, managing health, and the like.

By using the wearable terminal, data of daily activities, such as a body weight, a blood pressure, a heart rate, the number of steps, consumption calorie, sleep quality, and meal contents, can be recorded or measured. It is expected that a health state is monitored and utilized for early detection or prevention of a serious illness by using the wearable terminal.

A wearable terminal that can measure a superficial body temperature of a wrist, a heart rate, a blood pressure, and a blood oxygen concentration saturation is already commercially available. For example, in Apple Watch (registered trademark) Series 7 manufactured by Apple Inc., an electrocardiogram application and a blood oxygen wellness application are installed. In addition, on Jan. 5, 2021, Apple Inc. acquired a patent that can measure a blood pressure using an Apple Watch alone without using a cuff from the United States Patent and Trademark Office (USPTO).
https://iphone-mania.jp/news-338090/

In addition, The Wall Street Journal reported that there is a possibility that a body temperature measurement function is installed in an Apple Watch of 2022 model as early as possible.

https://iphone-mania.jp/news-394813/

As a wearable device capable of measuring a body temperature, Pham or the like developed a wearable device that integrates a heart rate, SpO₂, a physical activity level, a skin temperature, and an electrodermal response to estimate a core body temperature.
https://www.mdpi.com/1424-8220/20/3/855

In addition, Lin also developed a wearable device capable of measuring a body temperature of an actual runner.
https://www.mdpi.com/1424-8220/18/12/4347

The estimated temperature of Pham or the like reflects the core body temperature rather than the skin temperature, and in the estimated value of Lin or the like, there is no comparison with the skin temperature. In addition, both show only experimental data at 36° C. to 37° C., and it is unclear whether the data at approximately 40° C., which causes heat stroke, is correctly reflected. In addition, the device such as Pham is a device in which a cuff is attached to an upper arm, and the device such as Lin also needs to be attached to a wrist and an elbow, so that it is difficult to apply the devices to actual sports or labor. At present, a wearable device that can measure a core body temperature and can be applied to actual sports or labor is not developed.

In Glasgow Coma Scale (GCS) and Japan Coma Scale (JCS), a response to a call or a pain stimulation is used to evaluate a level of consciousness.
https://www.kango-roo.com/learning/3121/
https://www.kango-roo.com/learning/3082/

Apple Watch Series 7 has a fall detection function during a workout, which is a function of detecting a unique movement and an impact different from a normal case, such as a case of a fall while riding a bicycle or a case of a fall while standing, by a built-in accelerometer. If the built-in accelerometer determines that a consciousness disorder occurs by assuming a stop of moving, simple sleep will also be incorrectly determined as the consciousness disorder, and thus it can be said that a consciousness level cannot be monitored by a smart watch at the current situation.

In an intensive care unit of a hospital, monitoring of a movement of a thorax and measurement of a respiratory rate are performed, and the movement of the thorax cannot be monitored by the smart watch. In addition, with the smart watch, it is also difficult to monitor a blood test.

With progression of global warming and an increase in heat waves, heat stroke becomes an important public health issue. Despite the efforts of the government, local governments, and the media to raise awareness of heat stroke prevention, 71,317 people were transported by ambulance due to heat stroke nationwide in August 2020 (an increase of 6,305 people as compared to the same month of the previous year), and it is considered that further efforts for prevention are necessary in addition to the awareness through public relations and reporting based on Wet Bulb Globe Temperature (WBGT) (an indicator proposed in the United States in 1954 for the purpose of preventing heat stroke).
https://www.wbgt.env.go.jp/wbgt.php

In overseas, it is common to follow the Bouchama standard as follows, which is described in a review of N Engl J Med in 2002.
https://www.nejm.org/doi/full/10.1056/nejmra011089

Heat stroke corresponding to a severe case is defined as a severe case accompanied by a central nervous system disorder at a core body temperature of 40° C. or higher. It is described that Heat exhaustion is a mild to moderate state with various symptoms, and a core body temperature is often 37° C. to 40° C. In the Bouchama standard, it can be said that the core body temperature is a major factor in determining a severity.

In the following document, the present inventor showed that an in-hospital death rate is increased if Active Cooling is not performed in Heat stroke, and that the in-hospital death rate is not changed with intravenous drip alone in Heat exhaustion with multivariate analysis using various prognostic factors.

Kanda J, Nakahara S, Nakamura S, et al. “Association between active cooling and lower mortality among patients with heat stroke and heat exhaustion”. PLOS One. 2021; 16 (11): e0259441.
https://pubmed.ncbi.nlm.nih.gov/34788312/

The Bouchama standard is a world standard with clear evidence. Meanwhile, in the current wearable terminal, it is necessary to monitor the core body temperature and the consciousness disorder that cannot be measured.

In Japan, “Heat Stroke Classification 2015 of the Japan Association for Acute Medicine” is used as a diagnostic criterion for a severity of standard heat stroke.
https://www.kango-roo.com/work/1539/

In “Heat Stroke Classification 2015 of the Japan Association for Acute Medicine”, classification of I-degree to III-degree is performed according to clinical symptoms. The symptoms of III-degree which is the most severe, are a consciousness disorder, a liver and kidney disorder, and Disseminated Intravascular Coagulation (DIC), which correspond to Heat stroke. The symptoms of I-degree are dizziness, profuse sweating, fainting, and muscle pain, which correspond to Heat syncope and Heat cranp with leg cramp. The symptoms of II-degree are headache, vomiting, fatigue, a feeling of faintness, and a decrease in concentration or judgment (JCS is 1 or less), which correspond to Heat exhaustion.

In the severity classification in Japan, for the severe case, a blood test is used to use an element of multiple organ failure for determination of the severity. In addition, even in the mild case, it is necessary for a doctor to diagnose specific symptoms and determine the severity. Although “Heat Stroke Classification 2015 of the Japan Association for Acute Medicine” is the most widely used evaluation method in Japan, it is necessary to evaluate a consciousness disorder and to perform a blood test to determine the severity, and a specific diagnosis by the doctor is also required for the mild case, so that the evaluation method cannot be applied to the wearable terminal.

A J-ERATO score is configured with 6 items (a respiratory rate, a consciousness disorder, a systolic blood pressure, a heart rate, a body temperature, and an age) before arrival at a hospital, and a score of 0 or 1 is given to each of the items, and a total score is 0 to 6.
https://medit.tech/j-erato-score-app-released-2021/

The J-ERATO score is calculated based on data of approximately 3,000 cases of Heatstroke STUDY 2010 and 2012 conducted by the Japan Association for Acute Medicine, and the treatment content after hospitalization is not taken into consideration, and the evaluation items are hospitalization (denoted as “hospitalization rate”), entrance to the intensive care unit (ICU) (denoted as “ICU hospitalization rate”), and in-hospital death rate (denoted as “death rate”). In addition, the Japan Association for Acute Medicine created a “heat stroke diagnosis support application for a smartphone” based on the J-ERATO score. The application has a function of instructing first aid and displaying a map or a route to a nearby hospital, and there is no system that measures 6 items of the J-ERATO score in real time to prevent heat stroke.

The wearable terminal in the related art cannot monitor the consciousness disorder and the respiratory rate among 6 items of the J-ERATO score.

In “Mimamori Owl” which is a labor management/heat stroke management service provided by SOMPO Holdings, a heart rate and positional information of a worker are analyzed in real time by AI.
https://www.sompo-rc.co.jp/services_mimamorifukuro

From a website indicated by the URL described above, an AI diagnostic process is not clear, and it is considered that only the heart rate is extracted among 6 items of the J-ERATO score.

With the wearable terminal, it is not possible to measure a core body temperature of a wearer of the wearable terminal, so that in order to appropriately monitor the wearer of the wearable terminal by the wearable terminal, it is necessary to use another indicator instead of the core body temperature.

In any of the Bouchama standard, “Heat Stroke Classification 2015 of the Japan Association for Acute Medicine”, and the J-ERATO score, the degree of consciousness disorder is an evaluation item of a severity, so that in order to appropriately monitor the wearer of the wearable terminal by the wearable terminal, it is necessary to monitor the consciousness disorder of the wearer of the wearable terminal.

A superficial body temperature, a heart rate, a mean blood pressure, and a blood oxygen concentration saturation (SpO₂) of the wearer of the wearable terminal can be measured by the wearable terminal.

An acute physiology and chronic health evaluation (APACHE) II score is a prognosis prediction method created to objectively evaluate a severity of patients admitted to an intensive care unit, and is adopted in many facilities in Japan. In the APACHE II score, a predicted death rate is calculated by adding a score for an age and a chronic disease to 12 indicators of respiration, circulation, blood test values, and GCS.
https://www.fmu.ac.jp/home/masui/anesthesiologists/apache.html

As a result of intensive studies, the present inventor has found that, by causing a wearable terminal to output an alert for a consciousness disorder evaluation (for example, a sound, a vibration, a weak current that a wearer of the wearable terminal feels pain, and the like) used for evaluating the consciousness disorder of the wearer of the wearable terminal and detecting a reaction of the wearer of the wearable terminal (for example, detecting an input operation of the wearer of the wearable terminal to an operation unit of the wearable terminal, detecting a behavior of the wearer of the wearable terminal who shakes the wearable terminal by an acceleration sensor of the wearable terminal, and the like), the reaction of the wearer of the wearable terminal to the alert for evaluation of the consciousness disorder can be classified into any of “0: clear consciousness”, “1: orientation is maintained but consciousness is not clear”, “2: disorientation”, and “3: unable to say own name and date of birth”, “10: opening eyes in response to normal call”, “20: opening eyes in response to loud call, strong shake, or the like”, and “30: opening eyes with difficulty in response to call while continuing to apply pain stimulation”, “100: acting such as pushing away in response to pain”, “200: moving hands and feet or frown in response to pain stimulation”, and “300: no response at all to pain stimulation” of JCS.

On the other hand, if an alert for a consciousness disorder evaluation used for evaluating the consciousness disorder of the wearer of the wearable terminal is output excessively, there is a risk in which the wearer of the wearable terminal feels the alert for the consciousness disorder evaluation to be annoying.

In addition, if the evaluation of the consciousness disorder of the wearer of the wearable terminal is performed excessively, there is also a risk in which evaluation accuracy of the consciousness disorder of the wearer of the wearable terminal is reduced.

In view of the problems described above, in the embodiments of the monitoring system, the wearable terminal, the monitoring method, and the program according to the present invention, an output of an alert for a consciousness disorder evaluation is reduced at a necessary minimum, and a wearer of the wearable terminal is appropriately monitored in a necessary case based on a severity evaluation score including the evaluation of the consciousness disorder.

### First Embodiment

Hereinafter, embodiments of the monitoring system, the wearable terminal, the monitoring method, and the program according to the present invention will be described.

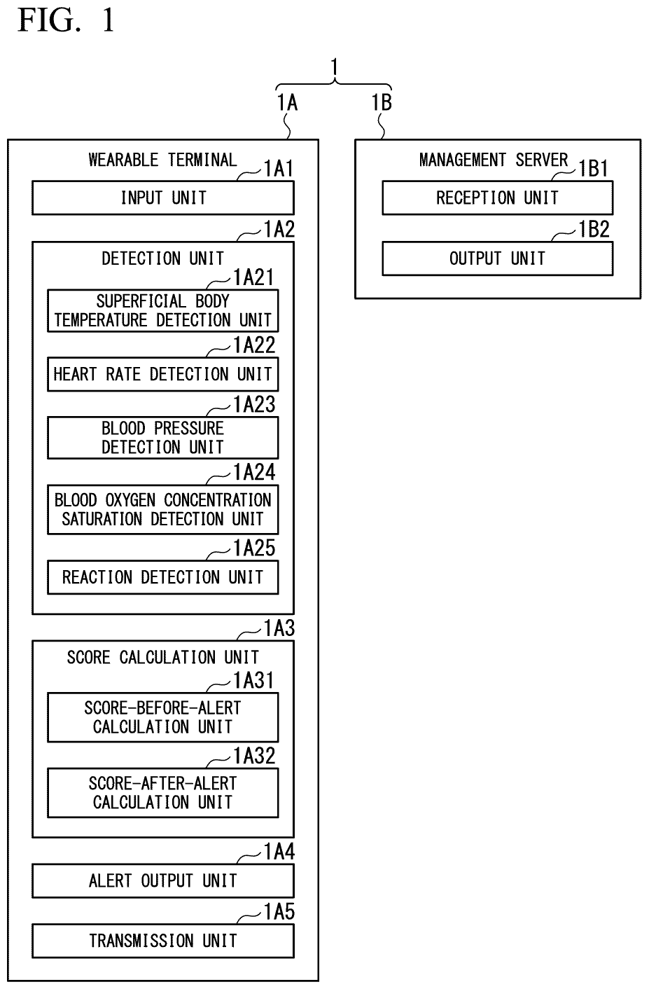

FIG. 1 is a diagram showing an example of a monitoring system 1 according to a first embodiment. The monitoring system 1 according to the first embodiment performs monitoring of a wearer of a wearable terminal 1A. In detail, the monitoring system 1 according to the first embodiment performs monitoring of a severity of heat stroke of the wearer of the wearable terminal 1A. That is, the monitoring system 1 according to the first embodiment is used for preventing occurrence of heat stroke or for preventing aggravation of heat stroke in the wearer of the wearable terminal 1A.

In the example shown in FIG. 1, the monitoring system 1 includes the wearable terminal 1A and a management server 1B. The wearable terminal 1A and the management server 1B are configured to be communicable with each other, for example, via a network or the like. The wearable terminal 1A is worn by a target person of monitoring by the monitoring system 1 according to the first embodiment. The wearable terminal 1A is, for example, a “watch type”, a “wristband type”, or the like.

In the example shown in FIG. 1, the wearable terminal 1A includes an input unit 1A1, a detection unit 1A2, a score calculation unit 1A3, an alert output unit 1A4, and a transmission unit 1A5.

The input unit 1A1 accepts an input operation of an age of the wearer of the wearable terminal 1A.

The detection unit 1A2 detects biological information of the wearer of the wearable terminal 1A.

The detection unit 1A2 includes a superficial body temperature detection unit 1A21, a heart rate detection unit 1A22, a blood pressure detection unit 1A23, a blood oxygen concentration saturation detection unit 1A24, and a reaction detection unit 1A25. The superficial body temperature detection unit 1A21 detects a superficial body temperature of the wearer of the wearable terminal 1A. The superficial body temperature detection unit 1A21 detects a superficial body temperature of the wearer of the wearable terminal 1A by using, for example, the technology described in P20 of the website indicated by the following URL.
https://www.soumu.go.jp/ict_skill/pdf/ict_skill_1_2.pdf

The heart rate detection unit 1A22 detects a heart rate of the wearer of the wearable terminal 1A. The heart rate detection unit 1A22 detects the heart rate of the wearer of the wearable terminal 1A by using, for example, the technology described in P19 and P20 of the website indicated by the URL described above.

The blood pressure detection unit 1A23 detects a mean blood pressure of the wearer of the wearable terminal 1A. The blood pressure detection unit 1A23 detects the mean blood pressure of the wearer of the wearable terminal 1A by using, for example, the technology disclosed in the website indicated by the following URL.
https://rentry.jp/note/blood-pressure-smart-watch/

The blood oxygen concentration saturation detection unit 1A24 detects a blood oxygen concentration saturation of the wearer of the wearable terminal 1A. The blood oxygen concentration saturation detection unit 1A24 detects the blood oxygen concentration saturation of the wearer of the wearable terminal 1A by using, for example, the technology disclosed in the website indicated by the following URL.
https://news.mynavi.jp/article/20150206-oximeter/
https://www.sankei.com/article/20201212-TQI2DCH7XBM35EJV6LPXLOP6ZU/
https://wired.jp/2020/12/06/how-to-use-blood-oxygen-data-on-apple-watch-garmin-fitbit-samsung/
https://www.watch.impress.co.jp/docs/review/minireview/1322495.html
https://monoist.atmarkit.co.jp/mn/articles/2010/13/news008.html

The reaction detection unit 1A25 detects a reaction of the wearer of the wearable terminal 1A to an alert for a consciousness disorder evaluation output by the alert output unit 1A4 (for example, a sound, a vibration, a weak current that the wearer of the wearable terminal feels pain, and the like). The reaction detection unit 1A25 detects the reaction of the wearer of the wearable terminal 1A to the alert for the consciousness disorder evaluation output by the alert output unit 1A4, by detecting, for example, an input operation of the wearer of the wearable terminal 1A to an operation unit (not shown) of the wearable terminal 1A, by an acceleration sensor (not shown) of the wearable terminal 1A detecting a shaking operation on the wearable terminal 1A by the wearer of the wearable terminal 1A, and the like.

That is, information on the wearer of the wearable terminal 1A detected by the detection unit 1A2 includes biological information of the wearer of the wearable terminal 1A, which is a superficial body temperature of the wearer of the wearable terminal 1A, a heart rate of the wearer of the wearable terminal 1A, a mean blood pressure of the wearer of the wearable terminal 1A, and a blood oxygen concentration saturation of the wearer of the wearable terminal 1A, and the reaction of the wearer of the wearable terminal 1A to the alert for the consciousness disorder evaluation output by the alert output unit 1A4.

The score calculation unit 1A3 calculates a severity evaluation score to be used for evaluating a severity of the wearer of the wearable terminal 1A, based on the biological information or the like of the wearer of the wearable terminal 1A detected by the detection unit 1A2. The score calculation unit 1A3 includes a score-before-alert calculation unit 1A31 and a score-after-alert calculation unit 1A32.

The score-before-alert calculation unit 1A31 calculates a severity evaluation score that does not include the evaluation of the consciousness disorder. Specifically, the score-before-alert calculation unit 1A31 calculates the severity evaluation score that does not include the evaluation of the consciousness disorder, based on the age of the wearer of the wearable terminal 1A accepted by the input unit 1A1, the superficial body temperature of the wearer of the wearable terminal 1A detected by the superficial body temperature detection unit 1A21, the heart rate of the wearer of the wearable terminal 1A detected by the heart rate detection unit 1A22, the mean blood pressure of the wearer of the wearable terminal 1A detected by the blood pressure detection unit 1A23, and the blood oxygen concentration saturation of the wearer of the wearable terminal 1A detected by the blood oxygen concentration saturation detection unit 1A24 (that is, based on the biological information of the wearer of the wearable terminal 1A).

The score-after-alert calculation unit 1A32 calculates a severity evaluation score including the evaluation of the consciousness disorder. Specifically, the score-after-alert calculation unit 1A32 calculates the severity evaluation score including the evaluation of the consciousness disorder, based on the age of the wearer of the wearable terminal 1A accepted by the input unit 1A1, the superficial body temperature of the wearer of the wearable terminal 1A detected by the superficial body temperature detection unit 1A21, the heart rate of the wearer of the wearable terminal 1A detected by the heart rate detection unit 1A22, the mean blood pressure of the wearer of the wearable terminal 1A detected by the blood pressure detection unit 1A23, the blood oxygen concentration saturation of the wearer of the wearable terminal 1A detected by the blood oxygen concentration saturation detection unit 1A24, and the reaction of the wearer of the wearable terminal 1A to the alert for the consciousness disorder evaluation detected by the reaction detection unit 1A25 (that is, based on the biological information of the wearer of the wearable terminal 1A and the reaction of the wearer of the wearable terminal 1A to the alert for the consciousness disorder evaluation).

The alert output unit 1A4 outputs an alert for a consciousness disorder evaluation (for example, a sound, a vibration, a weak current that the wearer of the wearable terminal 1A feels pain, and the like) used to evaluate the consciousness disorder of the wearer of the wearable terminal 1A.

The transmission unit 1A5 transmits the severity evaluation score calculated by the score calculation unit 1A3 to the management server 1B. That is, the transmission unit 1A5 transmits the severity evaluation score that does not include the evaluation of the consciousness disorder calculated by the score-before-alert calculation unit 1A31 of the score calculation unit 1A3 and the severity evaluation score including the evaluation of the consciousness disorder calculated by the score-after-alert calculation unit 1A32 of the score calculation unit 1A3 to the management server 1B.

In addition, the transmission unit 1A5 transmits the superficial body temperature, the heart rate, the mean blood pressure, and the blood oxygen concentration saturation of the wearer of the wearable terminal 1A (the biological information of the wearer of the wearable terminal 1A) used for the calculation of the severity evaluation score that does not include the evaluation of the consciousness disorder to the management server 1B.

Further, the transmission unit 1A5 transmits the superficial body temperature, the heart rate, the mean blood pressure, and the blood oxygen concentration saturation of the wearer of the wearable terminal 1A (the biological information of the wearer of the wearable terminal 1A), and the reaction of the wearer of the wearable terminal 1A to the alert for the consciousness disorder evaluation, which are used for the calculation of the severity evaluation score including the evaluation of the consciousness disorder, to the management server 1B.

The management server 1B includes a reception unit 1B1 and an output unit 1B2.

The reception unit 1B1 receives the severity evaluation score that does not include the evaluation of the consciousness disorder transmitted by the transmission unit 1A5 of the wearable terminal 1A, the biological information of the wearer of the wearable terminal 1A used for the calculation of the severity evaluation score that does not include the evaluation of the consciousness disorder, the severity evaluation score that includes the evaluation of the consciousness disorder, and the biological information of the wearer of the wearable terminal 1A and the reaction of the wearer of the wearable terminal 1A to the alert for the consciousness disorder evaluation, which are used for the calculation of the severity evaluation score that includes the evaluation of the consciousness disorder.

The output unit 1B2 outputs the severity evaluation score that does not include the evaluation of the consciousness disorder received by the reception unit 1B1, the biological information of the wearer of the wearable terminal 1A used for the calculation of the severity evaluation score that does not include the evaluation of the consciousness disorder, the severity evaluation score that includes the evaluation of the consciousness disorder, and the biological information of the wearer of the wearable terminal 1A and the reaction of the wearer of the wearable terminal 1A to the alert for the consciousness disorder evaluation, which are used for the calculation of the severity evaluation score that includes the evaluation of the consciousness disorder.

Therefore, a user of the management server 1B (a person who manages the wearer of the wearable terminal 1A) can check a severity or the like of heat stroke of the wearer of the wearable terminal 1A by checking the output from the output unit 1B2.

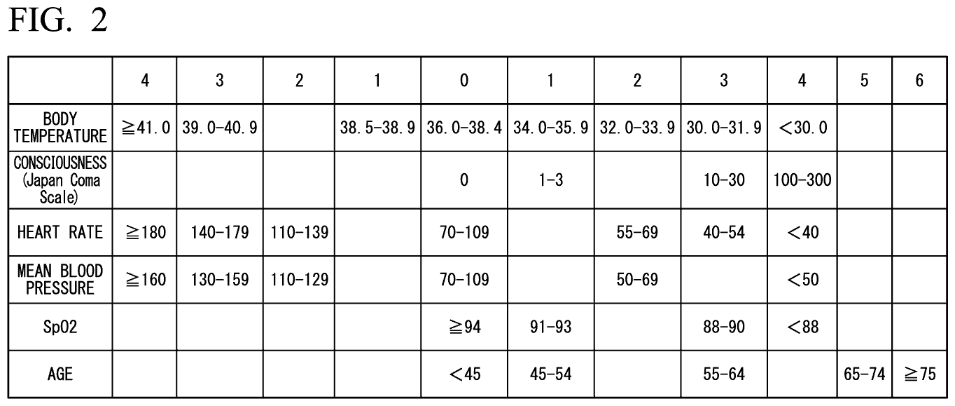

FIG. 2 is a diagram showing an example of a severity evaluation score calculated by the score calculation unit 1A3.

In the example shown in FIG. 2, the score calculation unit 1A3 calculates any of 0 points, 1 point, 3 points, 5 points, and 6 points as an age score, based on an age of the wearer of the wearable terminal 1A accepted by the input unit 1A1. Specifically, the score calculation unit 1A3 calculates 0 points as the age score in a case where the age of the wearer of the wearable terminal 1A is less than 45 years. The score calculation unit 1A3 calculates 1 point as the age score in a case where the age of the wearer of the wearable terminal 1A is 45 years or more and 54 years or less. The score calculation unit 1A3 calculates 3 points as the age score in a case where the age of the wearer of the wearable terminal 1A is 55 years or more and 64 years or less. The score calculation unit 1A3 calculates 5 points as the age score in a case where the age of the wearer of the wearable terminal 1A is 65 years or more and 74 years or less. The score calculation unit 1A3 calculates 6 points as the age score in a case where the age of the wearer of the wearable terminal 1A is 75 years or more.

The score calculation unit 1A3 calculates any of 0 points, 1 point, 2 points, 3 points, and 4 points as a superficial body temperature score, based on a superficial body temperature of the wearer of the wearable terminal 1A detected by the superficial body temperature detection unit 1A21. Specifically, the score calculation unit 1A3 calculates 0 points as the superficial body temperature score in a case where the superficial body temperature of the wearer of the wearable terminal 1A is 36.0° C. or higher and 38.4° C. or lower. The score calculation unit 1A3 calculates 1 point as the superficial body temperature score in a case where the superficial body temperature of the wearer of the wearable terminal 1A is 38.5° C. or higher and 38.9° C. or lower, or in a case where the superficial body temperature of the wearer of the wearable terminal 1A is 34.0° C. or higher and 35.9° C. or lower. The score calculation unit 1A3 calculates 2 points as the superficial body temperature score in a case where the superficial body temperature of the wearer of the wearable terminal 1A is 32.0° C. or higher and 33.9° C. or lower. The score calculation unit 1A3 calculates 3 points as the superficial body temperature score in a case where the superficial body temperature of the wearer of the wearable terminal 1A is 39.0° C. or higher and 40.9° C. or lower, or in a case where the superficial body temperature of the wearer of the wearable terminal 1A is 30.0° C. or higher and 31.9° C. or lower. The score calculation unit 1A3 calculates 4 points as the superficial body temperature score in a case where the superficial body temperature of the wearer of the wearable terminal 1A is 41.0° C. or higher or in a case where the superficial body temperature of the wearer of the wearable terminal 1A is less than 30.0° C.

The score calculation unit 1A3 calculates any of 0 points, 2 points, 3 points, and 4 points as a heart rate score, based on a heart rate of the wearer of the wearable terminal 1A detected by the heart rate detection unit 1A22. Specifically, the score calculation unit 1A3 calculates 0 points as the heart rate score in a case where the heart rate [beats/min] of the wearer of the wearable terminal 1A is 70 or more and 109 or less. The score calculation unit 1A3 calculates 2 points as the heart rate score in a case where the heart rate [beats/min] of the wearer of the wearable terminal 1A is 110 or more and 139 or less or in a case where the heart rate [beats/min] of the wearer of the wearable terminal 1A is 55 or more and 69 or less. The score calculation unit 1A3 calculates 3 points as the heart rate score in a case where the heart rate [beats/min] of the wearer of the wearable terminal 1A is 140 or more and 179 or less or in a case where the heart rate [beats/min] of the wearer of the wearable terminal 1A is 40 or more and 54 or less. The score calculation unit 1A3 calculates 4 points as the heart rate score in a case where the heart rate [beats/min] of the wearer of the wearable terminal 1A is 180 or more or in a case where the heart rate [beats/min] of the wearer of the wearable terminal 1A is less than 40.

The score calculation unit 1A3 calculates any of 0 points, 2 points, 3 points, and 4 points as a mean blood pressure score, based on a mean blood pressure of the wearer of the wearable terminal 1A detected by the blood pressure detection unit 1A23. Specifically, the score calculation unit 1A3 calculates 0 points as the mean blood pressure score in a case where the mean blood pressure [mmHg] of the wearer of the wearable terminal 1A is 70 or more and 109 or less. The score calculation unit 1A3 calculates 2 points as the mean blood pressure score in a case where the mean blood pressure [mmHg] of the wearer of the wearable terminal 1A is 110 or more and 129 or less, or in a case where the mean blood pressure [mmHg] of the wearer of the wearable terminal 1A is 50 or more and 69 or less. The score calculation unit 1A3 calculates 3 points as the mean blood pressure score in a case where the mean blood pressure [mmHg] of the wearer of the wearable terminal 1A is 130 or more and 159 or less. The score calculation unit 1A3 calculates 4 points as the mean blood pressure score in a case where the mean blood pressure [mmHg] of the wearer of the wearable terminal 1A is 160 or more or in a case where the mean blood pressure [mmHg] of the wearer of the wearable terminal 1A is less than 50.

The score calculation unit 1A3 calculates any of 0 points, 1 point, 3 points, and 4 points as a blood oxygen concentration saturation score, based on a blood oxygen concentration saturation (SpO₂) of the wearer of the wearable terminal 1A detected by the blood oxygen concentration saturation detection unit 1A24. Specifically, the score calculation unit 1A3 calculates 0 points as the blood oxygen concentration saturation score in a case where the blood oxygen concentration saturation [%] of the wearer of the wearable terminal 1A is 94% or more. The score calculation unit 1A3 calculates 1 point as the blood oxygen concentration saturation score in a case where the blood oxygen concentration saturation [%] of the wearer of the wearable terminal 1A is 91 or more and 93 or less. The score calculation unit 1A3 calculates 3 points as the blood oxygen concentration saturation score in a case where the blood oxygen concentration saturation [%] of the wearer of the wearable terminal 1A is 88 or more and 90 or less. The score calculation unit 1A3 calculates 4 points as the blood oxygen concentration saturation score in a case where the blood oxygen concentration saturation [%] of the wearer of the wearable terminal 1A is less than 88.

The score calculation unit 1A3 calculates any of 0 points, 1 point, 3 points, and 4 points as a consciousness disorder score, based on a reaction of the wearer of the wearable terminal 1A to an alert for a consciousness disorder evaluation detected by the reaction detection unit 1A25.

Specifically, the score calculation unit 1A3 calculates 0 points as the consciousness disorder score in a case where the reaction of the wearer of the wearable terminal 1A to the alert for the consciousness disorder evaluation corresponds to “0: clear consciousness” of JCS.

In a first example of the monitoring system 1 according to the first embodiment, in a case where a time from a time point at which the alert output unit 1A4 outputs the alert for the consciousness disorder evaluation to a time point at which the reaction detection unit 1A25 detects the reaction of the wearer of the wearable terminal 1A is less than a threshold value T1 [seconds], the score calculation unit 1A3 calculates the consciousness disorder score “0 points” with which the reaction of the wearer of the wearable terminal 1A to the alert for the consciousness disorder evaluation corresponds to “0: clear consciousness” of JCS. The value of the threshold value T1 [seconds] is set in advance in an experiment or the like.

In a second example of the monitoring system 1 according to the first embodiment, in a case where an acceleration of a motion of the wearer of the wearable terminal 1A detected by the reaction detection unit 1A25 (acceleration sensor) is equal to or more than a threshold value G1, the score calculation unit 1A3 calculates the consciousness disorder score “0 points” with which the reaction of the wearer of the wearable terminal 1A to the alert for the consciousness disorder evaluation corresponds to “0: clear consciousness” of JCS. The value of the threshold value G1 is set in advance in an experiment or the like.

In the example shown in FIG. 2, the score calculation unit 1A3 calculates 1 point as the consciousness disorder score in a case where the reaction of the wearer of the wearable terminal 1A to the alert for the consciousness disorder evaluation corresponds to “1: orientation is maintained but consciousness is not clear”, “2: disorientation”, or “3: unable to say own name and date of birth” of JCS.

In the first example of the monitoring system 1 according to the first embodiment, in a case where the time from the time point at which the alert output unit 1A4 outputs the alert for the consciousness disorder evaluation to the time point at which the reaction detection unit 1A25 detects the reaction of the wearer of the wearable terminal 1A is equal to or more than the threshold value T1 [seconds] and less than a threshold value T2 (>T1) [seconds], the score calculation unit 1A3 calculates the consciousness disorder score “1 point” with which the reaction of the wearer of the wearable terminal 1A to the alert for the consciousness disorder evaluation corresponds to “1: orientation is maintained but consciousness is not clear”, “2: disorientation”, or “3: unable to say own name and date of birth” of JCS. The value of the threshold value T2 [seconds] is set in advance in an experiment or the like.

In the example shown in FIG. 2, the score calculation unit 1A3 calculates 3 points as the consciousness disorder score in a case where the reaction of the wearer of the wearable terminal 1A to the alert for the consciousness disorder evaluation corresponds to “10: opening eyes in response to normal call”, “20: opening eyes in response to loud call, strong shake, or the like”, or “30: opening eyes with difficulty in response to call while continuing to apply pain stimulation” of JCS.

In the first example of the monitoring system 1 according to the first embodiment and in the second example of the monitoring system 1 according to the first embodiment, in a case where the reaction detection unit 1A25 cannot detect and observe the reaction of the wearer of the wearable terminal 1A up to the threshold value T2 [seconds] and in a case where the acceleration of the motion of the wearer of the wearable terminal 1A detected by the reaction detection unit 1A25 (acceleration sensor) is equal to or more than the threshold value G1, the score calculation unit 1A3 calculates the consciousness disorder score “3 points” with which the reaction of the wearer of the wearable terminal 1A to the alert for the consciousness disorder evaluation corresponds to “10: opening eyes in response to normal call”, “20: opening eyes in response to loud call, strong shake, or the like”, or “30: opening eyes with difficulty in response to call while continuing to apply pain stimulation” of JCS. The value of the threshold value G1 is set in advance in an experiment or the like.

In the example shown in FIG. 2, the score calculation unit 1A3 calculates 4 points as the consciousness disorder score in a case where the reaction of the wearer of the wearable terminal 1A to the alert for the consciousness disorder evaluation corresponds to “100: acting such as pushing away in response to pain”, “200: moving hands and feet or frown in response to pain stimulation”, or “300: no response at all to pain stimulation” of JCS.

In the first example of the monitoring system 1 according to the first embodiment, in a case where the reaction detection unit 1A25 cannot detect and observe the reaction of the wearer of the wearable terminal 1A within the threshold value T2 [seconds] from the time point at which the alert output unit 1A4 outputs the alert for the consciousness disorder evaluation, and the acceleration of the motion of the wearer of the wearable terminal 1A detected by the reaction detection unit 1A25 (acceleration sensor) is less than the threshold value G1, the score calculation unit 1A3 calculates the consciousness disorder score “4 points” with which the reaction of the wearer of the wearable terminal 1A to the alert for the consciousness disorder evaluation corresponds to “100: acting such as pushing away in response to pain”, “200: moving hands and feet or frown in response to pain stimulation”, or “300: no response at all to pain stimulation” of JCS.

In the examples shown in FIGS. 1 and 2, in a first mode (normal mode) of the monitoring system 1 according to the first embodiment, the alert output unit 1A4 does not output an alert for a consciousness disorder evaluation to reduce the output of the alert for the consciousness disorder evaluation by the alert output unit 1A4 at a necessary minimum.

The detection unit 1A2 detects biological information of the wearer of the wearable terminal 1A in a state in which the alert output unit 1A4 does not output the alert for the consciousness disorder evaluation (first mode). Specifically, the superficial body temperature detection unit 1A21 of the detection unit 1A2 detects a superficial body temperature of the wearer of the wearable terminal 1A, the heart rate detection unit 1A22 detects a heart rate of the wearer of the wearable terminal 1A, the blood pressure detection unit 1A23 detects a mean blood pressure of the wearer of the wearable terminal 1A, and the blood oxygen concentration saturation detection unit 1A24 detects a blood oxygen concentration saturation of the wearer of the wearable terminal 1A. The score-before-alert calculation unit 1A31 of the score calculation unit 1A3 calculates a severity evaluation score (severity evaluation score of 5 items) that does not include an evaluation of a consciousness disorder, based on an age of the wearer of the wearable terminal 1A accepted by the input unit 1A1, the superficial body temperature of the wearer of the wearable terminal 1A detected by the superficial body temperature detection unit 1A21, the heart rate of the wearer of the wearable terminal 1A detected by the heart rate detection unit 1A22, the mean blood pressure of the wearer of the wearable terminal 1A detected by the blood pressure detection unit 1A23, and the blood oxygen concentration saturation of the wearer of the wearable terminal 1A detected by the blood oxygen concentration saturation detection unit 1A24. Specifically, the score-before-alert calculation unit 1A31 calculates the severity evaluation score (severity evaluation score of 5 items) that does not include the evaluation of the consciousness disorder by adding the age score, the superficial body temperature score, the heart rate score, the mean blood pressure score, and the blood oxygen concentration saturation score.

In the example shown in FIGS. 1 and 2, in a case where the severity evaluation score (severity evaluation score of 5 items) that does not include the evaluation of the consciousness disorder calculated by the score-before-alert calculation unit 1A31 of the score calculation unit 1A3 is equal to or more than a threshold value (specifically, 3 points), the monitoring system 1 of the first embodiment is switched from the first mode to a second mode.

In the examples shown in FIGS. 1 and 2, the monitoring system 1 according to the first embodiment is switched from the first mode to the second mode in a case where the severity evaluation score that does not include the evaluation of the consciousness disorder is 3 points or more, and in other examples, the monitoring system 1 according to the first embodiment may be switched from the first mode to the second mode in a case where the severity evaluation score that does not include the evaluation of the consciousness disorder is a score equal to or more than a score (for example, 5 points) other than 3 points.

In the example shown in FIGS. 1 and 2, in the second mode of the monitoring system 1 according to the first embodiment, the alert output unit 1A4 outputs the alert for the consciousness disorder evaluation.

The detection unit 1A2 detects the biological information of the wearer of the wearable terminal 1A and the reaction of the wearer of the wearable terminal 1A to the alert for the consciousness disorder evaluation output by the alert output unit 1A4 in a state in which the alert output unit 1A4 outputs the alert for the consciousness disorder evaluation (second mode). Specifically, the superficial body temperature detection unit 1A21 of the detection unit 1A2 detects the superficial body temperature of the wearer of the wearable terminal 1A, the heart rate detection unit 1A22 detects the heart rate of the wearer of the wearable terminal 1A, the blood pressure detection unit 1A23 detects the mean blood pressure of the wearer of the wearable terminal 1A, the blood oxygen concentration saturation detection unit 1A24 detects the blood oxygen concentration saturation of the wearer of the wearable terminal 1A, and the reaction detection unit 1A25 detects the reaction of the wearer of the wearable terminal 1A to the alert for the consciousness disorder evaluation output by the alert output unit 1A4.

The score calculation unit 1A3 calculates a severity evaluation score (severity evaluation score of 6 items) including the evaluation of the consciousness disorder, based on the biological information or the like of the wearer of the wearable terminal 1A detected by the detection unit 1A2 and the reaction of the wearer of the wearable terminal 1A to the alert for the consciousness disorder evaluation output by the alert output unit 1A4.

Specifically, the score-after-alert calculation unit 1A32 of the score calculation unit 1A3 calculates the severity evaluation score (severity evaluation score of 6 items) including the evaluation of the consciousness disorder, based on the age of the wearer of the wearable terminal 1A accepted by the input unit 1A1, the superficial body temperature of the wearer of the wearable terminal 1A detected by the superficial body temperature detection unit 1A21, the heart rate of the wearer of the wearable terminal 1A detected by the heart rate detection unit 1A22, the mean blood pressure of the wearer of the wearable terminal 1A detected by the blood pressure detection unit 1A23, the blood oxygen concentration saturation of the wearer of the wearable terminal 1A detected by the blood oxygen concentration saturation detection unit 1A24, and the reaction of the wearer of the wearable terminal 1A to the alert for the consciousness disorder evaluation detected by the reaction detection unit 1A25. Specifically, the score-after-alert calculation unit 1A32 calculates the severity evaluation score (severity evaluation score of 6 items) including the evaluation of the consciousness disorder, by adding the age score, the superficial body temperature score, the heart rate score, the mean blood pressure score, the blood oxygen concentration saturation score, and the consciousness disorder score.

In the examples shown in FIGS. 1 and 2, in the second mode of the monitoring system 1 according to the first embodiment, the transmission unit 1A5 transmits the severity evaluation score (severity evaluation score of 6 items) including the evaluation of the consciousness disorder calculated by the score-after-alert calculation unit 1A32 of the score calculation unit 1A3, the superficial body temperature of the wearer of the wearable terminal 1A detected by the superficial body temperature detection unit 1A21, the heart rate of the wearer of the wearable terminal 1A detected by the heart rate detection unit 1A22, the mean blood pressure of the wearer of the wearable terminal 1A detected by the blood pressure detection unit 1A23, the blood oxygen concentration saturation of the wearer of the wearable terminal 1A detected by the blood oxygen concentration saturation detection unit 1A24, and the reaction of the wearer of the wearable terminal 1A to the alert for the consciousness disorder evaluation detected by the reaction detection unit 1A25, to the management server 1B.

The reception unit 1B1 of the management server 1B receives the severity evaluation score (severity evaluation score of 6 items) including the evaluation of the consciousness disorder, the superficial body temperature of the wearer of the wearable terminal 1A, the heart rate of the wearer of the wearable terminal 1A, the mean blood pressure of the wearer of the wearable terminal 1A, the blood oxygen concentration saturation of the wearer of the wearable terminal 1A, and the reaction of the wearer of the wearable terminal 1A to the alert for the consciousness disorder evaluation, which are transmitted by the transmission unit 1A5 of the wearable terminal 1A.

The output unit 1B2 of the management server 1B outputs the severity evaluation score (severity evaluation score of 6 items) including the evaluation of the consciousness disorder, the superficial body temperature of the wearer of the wearable terminal 1A, the heart rate of the wearer of the wearable terminal 1A, the mean blood pressure of the wearer of the wearable terminal 1A, the blood oxygen concentration saturation of the wearer of the wearable terminal 1A, and the reaction of the wearer of the wearable terminal 1A to the alert for the consciousness disorder evaluation, which are received by the reception unit 1B1.

Therefore, the user of the management server 1B (the person who manages the wearer of the wearable terminal 1A) can check a severity of heat stroke of the wearer of the wearable terminal 1A, by checking the severity evaluation score (severity evaluation score of 6 items) including the evaluation of the consciousness disorder, the superficial body temperature of the wearer of the wearable terminal 1A, the heart rate of the wearer of the wearable terminal 1A, the mean blood pressure of the wearer of the wearable terminal 1A, the blood oxygen concentration saturation of the wearer of the wearable terminal 1A, and the reaction of the wearer of the wearable terminal 1A to the alert for the consciousness disorder evaluation, which are output by the output unit 1B2.

That is, with the monitoring system 1 according to the first embodiment, the wearer of the wearable terminal 1A can be appropriately monitored based on the severity evaluation score including the evaluation of the consciousness disorder in a necessary case while reducing the output of the alert for the consciousness disorder evaluation at a necessary minimum.

In addition, with the monitoring system 1 according to the first embodiment, it is expected that the alert for the consciousness disorder evaluation leads to a change in behavior (turning on a cooler, taking a rest from work or sports, and the like) even in the mild case (incipient case).

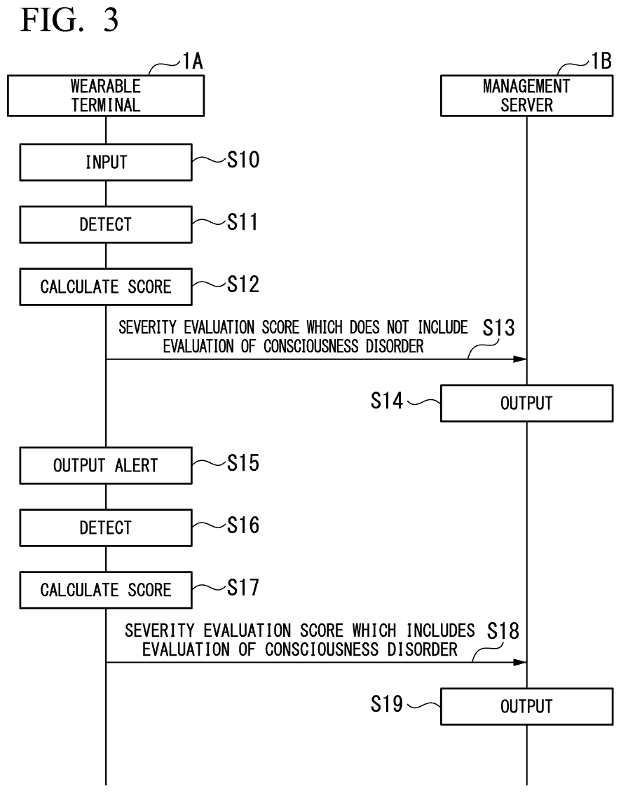

FIG. 3 is a sequence diagram showing an example of a process executed in the monitoring system 1 according to the first embodiment.

In the example shown in FIG. 3, in step S10, the input unit 1A1 of the wearable terminal 1A accepts an input operation of an age of a wearer of the wearable terminal 1A.

Next, in step S11, the detection unit 1A2 of the wearable terminal 1A detects biological information of the wearer of the wearable terminal 1A (that does not include a reaction of the wearer of the wearable terminal 1A to an alert for a consciousness disorder evaluation output by the alert output unit 1A4). Specifically, the superficial body temperature detection unit 1A21 of the detection unit 1A2 detects a superficial body temperature of the wearer of the wearable terminal 1A, the heart rate detection unit 1A22 detects a heart rate of the wearer of the wearable terminal 1A, the blood pressure detection unit 1A23 detects a mean blood pressure of the wearer of the wearable terminal 1A, and the blood oxygen concentration saturation detection unit 1A24 detects a blood oxygen concentration saturation of the wearer of the wearable terminal 1A.

Next, in step S12, the score calculation unit 1A3 of the wearable terminal 1A calculates a severity evaluation score (severity evaluation score that does not include an evaluation of a consciousness disorder) used for an evaluation of a severity of the wearer of the wearable terminal 1A, based on the biological information or the like of the wearer of the wearable terminal 1A detected in step S11.

Next, in step S13, the transmission unit 1A5 of the wearable terminal 1A transmits the severity evaluation score (severity evaluation score that does not include the evaluation of the consciousness disorder) calculated in step S12 and the biological information of the wearer of the wearable terminal 1A to the management server 1B, and the output unit 1B2 of the management server 1B receives the severity evaluation score (severity evaluation score that does not include the evaluation of the consciousness disorder) and the biological information of the wearer of the wearable terminal 1A.

Next, in step S14, the output unit 1B2 of the management server 1B outputs the severity evaluation score (severity evaluation score that does not include the evaluation of the consciousness disorder) and the biological information of the wearer of the wearable terminal 1A, which are received in step S13.

In the example shown in FIG. 3, in step S15, the severity evaluation score that does not include the evaluation of the consciousness disorder is equal to or more than a threshold value (for example, 3 points), and the alert output unit 1A4 of the wearable terminal 1A outputs an alert for a consciousness disorder evaluation.

Next, in step S16, the detection unit 1A2 of the wearable terminal 1A detects biological information of the wearer of the wearable terminal 1A and a reaction of the wearer of the wearable terminal 1A to the alert for the consciousness disorder evaluation output by the alert output unit 1A4, in a state in which the alert output unit 1A4 outputs the alert for the consciousness disorder evaluation.

Next, in step S17, the score calculation unit 1A3 of the wearable terminal 1A calculates a severity evaluation score including the evaluation of the consciousness disorder, based on the biological information or the like of the wearer of the wearable terminal 1A and the reaction of the wearer of the wearable terminal 1A to the alert for the consciousness disorder evaluation output by the alert output unit 1A4, which are detected in step S16.

Next, in step S18, the transmission unit 1A5 of the wearable terminal 1A transmits the severity evaluation score including the evaluation of the consciousness disorder calculated in step S17, the biological information of the wearer of the wearable terminal 1A, and the reaction of the wearer of the wearable terminal 1A to the alert for the consciousness disorder evaluation to the management server 1B, and the output unit 1B2 of the management server 1B receives the severity evaluation score including the evaluation of the consciousness disorder, the biological information of the wearer of the wearable terminal 1A, and the reaction of the wearer of the wearable terminal 1A to the alert for the consciousness disorder evaluation.

Next, in step S19, the output unit 1B2 of the management server 1B outputs the severity evaluation score including the evaluation of the consciousness disorder, the biological information of the wearer of the wearable terminal 1A, and the reaction of the wearer of the wearable terminal 1A to the alert for the consciousness disorder evaluation, which are received in step S18.

### EXAMPLES

The present inventor adopted occurrence of Heat stroke (core body temperature of 40° C. or higher and GCS of 8 or lower) at a time of a patient's visit to a hospital as an evaluation item of heat stroke. In a case where the core body temperature is 40° C. or higher and the GCS is 8 or lower, Active Cooling (active cooling on whole body) is important for improving the outcome, and thus it can be said that it is necessary to transport the patient to the hospital as soon as possible in this case.

The present inventor evaluated a consciousness level of a wearer of the wearable terminal 1A based on a reaction of the wearer of the wearable terminal 1A to an alert for a consciousness disorder evaluation output in a case where a severity evaluation score that does not include an evaluation of a consciousness disorder is 3 points or more.

The present inventor used, as the evaluation items, an age of the wearer of the wearable terminal 1A, a superficial body temperature of the wearer of the wearable terminal 1A, a heart rate of the wearer of the wearable terminal 1A, a mean blood pressure of the wearer of the wearable terminal 1A, a blood oxygen concentration saturation of the wearer of the wearable terminal 1A, and a reaction of the wearer of the wearable terminal 1A to the alert for the consciousness disorder evaluation output by the alert output unit 1A4.

The present inventor calculated the severity evaluation score (score-before-alert) that does not include the evaluation of the consciousness disorder, based on the age of the wearer of the wearable terminal 1A, the superficial body temperature of the wearer of the wearable terminal 1A, the heart rate of the wearer of the wearable terminal 1A, the mean blood pressure of the wearer of the wearable terminal 1A, and the blood oxygen concentration saturation of the wearer of the wearable terminal 1A, and calculated a severity evaluation score (score-after-alert) that includes the evaluation of the consciousness disorder, based on the age of the wearer of the wearable terminal 1A, the superficial body temperature of the wearer of the wearable terminal 1A, the heart rate of the wearer of the wearable terminal 1A, the mean blood pressure of the wearer of the wearable terminal 1A, the blood oxygen concentration saturation of the wearer of the wearable terminal 1A, and the reaction of the wearer of the wearable terminal 1A to the alert for the consciousness disorder evaluation.

In Heat Stroke STUDY conducted by the Japan Association for Acute Medicine from 2008 to 2020, the present inventor targeted 1089 cases (excluding cases of cardiopulmonary arrest (CPA) in front of the hospital) and cases of oxygen administration) in which information before a hospital and information at a time of visiting the hospital was clear for the severity evaluation of heat stroke.

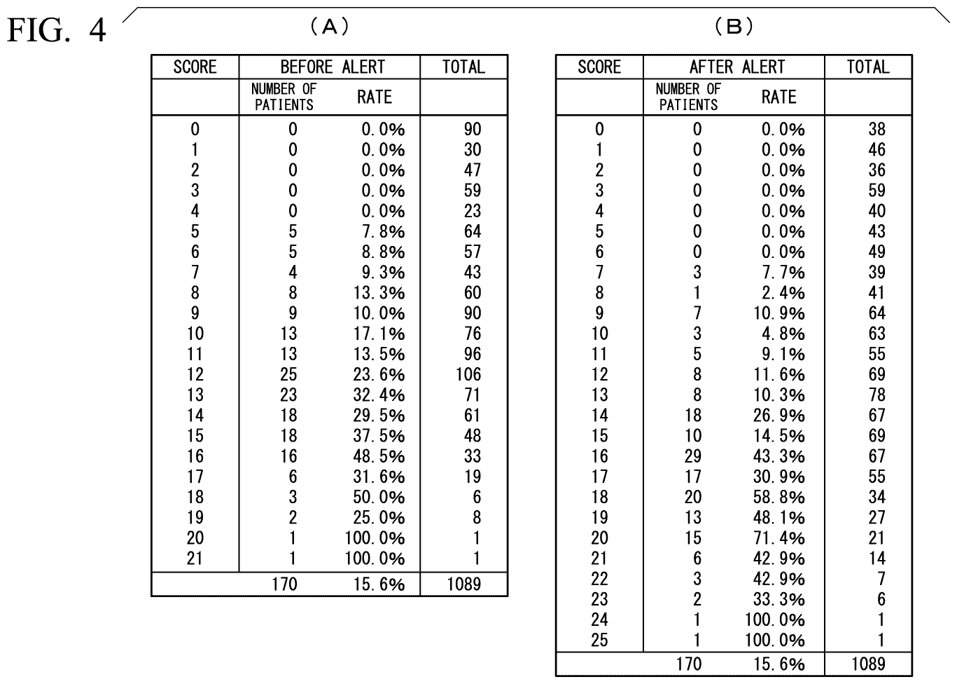

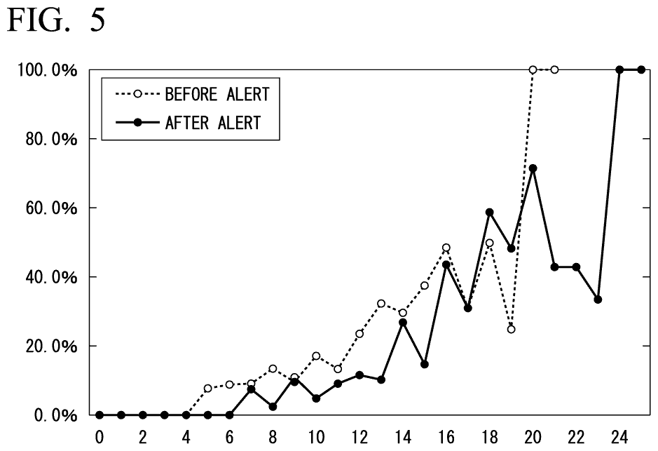

FIG. 4 is a diagram showing a relationship between a severity evaluation score, the number of patients, an occurrence rate of Heat stroke (core body temperature of 40° C. or higher and GCS of 8 or lower), and the number of cases in the example of the monitoring system 1 according to the first embodiment. Specifically, (A) of FIG. 4 shows a relationship between a score-before-alert and the number of patients or the like in the example of the monitoring system 1 according to the first embodiment, and (B) of FIG. 4 shows a relationship between a score-after-alert and the number of patients or the like in the example of the monitoring system 1 according to the first embodiment. FIG. 5 is a diagram comparing a relationship between the severity evaluation score (horizontal axis) and the occurrence rate of Heat stroke (core body temperature of 40° C. or higher and GCS of 8 or lower) (vertical axis) in the example of the monitoring system 1 according to the first embodiment with the score-before-alert and the score-after-alert.

The present inventor studied a threshold value (point of a severity evaluation score that does not include an evaluation of a consciousness disorder) at which the monitoring system 1 is to be switched from the first mode to the second mode.

In a case where the threshold value at which the monitoring system 1 is to be switched from the first mode to the second mode is set to 3 points, the occurrence rate is 0.0% only before the alert. In the consciousness disorder of 4 points, the score after the alert becomes 7 points and the occurrence rate is 7.7%. It is considered that the threshold value “3 points” is an appropriate timing for promoting an early change in behavior.

In a case where the threshold value at which the monitoring system 1 is to be switched from the first mode to the second mode is set to 5 points, the occurrence rate is 7.8% only before the alert. In a case where there is no consciousness disorder, the score after the alert is 5 points and the occurrence rate is 0.0%, and in a case where the consciousness disorder has 4 points, the score after the alert becomes 9 points and the occurrence rate is 10.9%. It is a general rule to prompt a change in behavior again and to transport the person to a hospital in a case where the person has a consciousness disorder, and there is also a possibility that the person can be watched in a medical room or the like.

In a case where the threshold value at which the monitoring system 1 is to be switched from the first mode to the second mode is set to 8 points, the occurrence rate before the alert is 13.3%. In a case where there is no the consciousness disorder, the score after the alert is 8 points and the occurrence rate is 2.4%, and in a case where there is a consciousness disorder, the score after the alert becomes 12 points and the occurrence rate is 11.6%. In a case where there is no the consciousness disorder, it is also possible to promote a change in behavior. In a case where there is a consciousness disorder, the general rule is to transport the patient to the hospital, and it is difficult to observe the patient in a case where the patient is not actively treated in a medical room or the like.

In a case where the threshold value at which the monitoring system 1 is to be switched from the first mode to the second mode is set to 10 points, the occurrence rate before the alert is 17.1%. In a case where there is no the consciousness disorder, the score after the alert is 10 points and the occurrence rate is 4.8%, and in a case where there is a consciousness disorder, the score after the alert becomes 14 points and the occurrence rate is 26.9%. It is also possible to promote a change in behavior, and in a case where there is a consciousness disorder, the transport to the hospital is at an appropriate level rather than the transport to a medical room.

In a case where the threshold value at which the monitoring system 1 is to be switched from the first mode to the second mode is set to 12 points, the occurrence rate before the alert is 23.6%. In a case where there is no the consciousness disorder, the score after the alert is 12 points and the occurrence rate is 11.6%, and in a case where there is a consciousness disorder, the score after the alert becomes 16 points and the occurrence rate is 43.3%. It can be said that, even in a case where there is no the consciousness disorder, the remote watching should be stopped and a direct check is to be performed. In a case of a consciousness disorder, early transportation to the hospital is necessary.

In a case where the threshold value at which the monitoring system 1 is to be switched from the first mode to the second mode is set to 14 points, the occurrence rate is 29.5% only before the alert. Even in a case where there is no consciousness disorder, the score after the alert is 14 points and the occurrence rate is 26.9%. In a case where there is a consciousness disorder, the score after the alert is 18 points and the occurrence rate is 58.8%. The level is desirable for transportation to the hospital even in a case where there is no consciousness disorder. In a case where there is a consciousness disorder, a probability of being a target of a life-saving aid is high.

### Second Embodiment

Hereinafter, a second embodiment of the monitoring system, the wearable terminal, the monitoring method, and the program according to the present invention will be described.

The monitoring system 1 according to the second embodiment is configured in the same manner as the monitoring system 1 according to the first embodiment described above, except for the following points. Therefore, with the monitoring system 1 of the second embodiment, the same effects as those of the monitoring system 1 of the first embodiment described above can be obtained, except for the following points.

The monitoring system 1 according to the second embodiment is configured in the same manner as the monitoring system 1 according to the first embodiment shown in FIG. 1. The monitoring system 1 according to the second embodiment performs monitoring on a wearer of the wearable terminal 1A, in the same manner as the monitoring system 1 according to the first embodiment. In detail, the monitoring system 1 according to the second embodiment performs monitoring of a disease severity other than heat stroke of the wearer of the wearable terminal 1A. That is, the monitoring system 1 according to the second embodiment is used for preventing aggravation of a disease other than heat stroke of the wearer of the wearable terminal 1A.

In the monitoring system 1 according to the second embodiment, a user (a person who manages the wearer of the wearable terminal 1A) of the management server 1B can check the disease severity other than heat stroke of the wearer of the wearable terminal 1A, by checking a severity evaluation score output by the output unit 1B2.

In the example of the monitoring system 1 according to the second embodiment, in the same manner as in the example shown in FIG. 2 of the monitoring system 1 according to the first embodiment, a severity evaluation score is calculated by the score calculation unit 1A3.

That is, even with the monitoring system 1 according to the second embodiment, the wearer of the wearable terminal 1A can be appropriately monitored based on the severity evaluation score including an evaluation of a consciousness disorder in a necessary case while reducing an output of an alert for the consciousness disorder evaluation at a necessary minimum.

In the monitoring system 1 according to the second embodiment, the same process as the processing shown in FIG. 3 executed in the monitoring system 1 according to the first embodiment is executed.

### EXAMPLES

The present inventor applied the monitoring system 1 according to the second embodiment to 1,726 patients who were hospitalized in the advanced medical care and emergency center of Teikyo University School of Medicine, and compared a score before a hospital and a severity after visiting the hospital. A severe case after visiting the hospital is defined as a case in which tracheal intubation is executed or a case in which the outcome at a time of leaving the hospital is death or a serious sequelae since there are cases in which the tracheal intubation is not executed at a request of the patient or family even in severe cases.

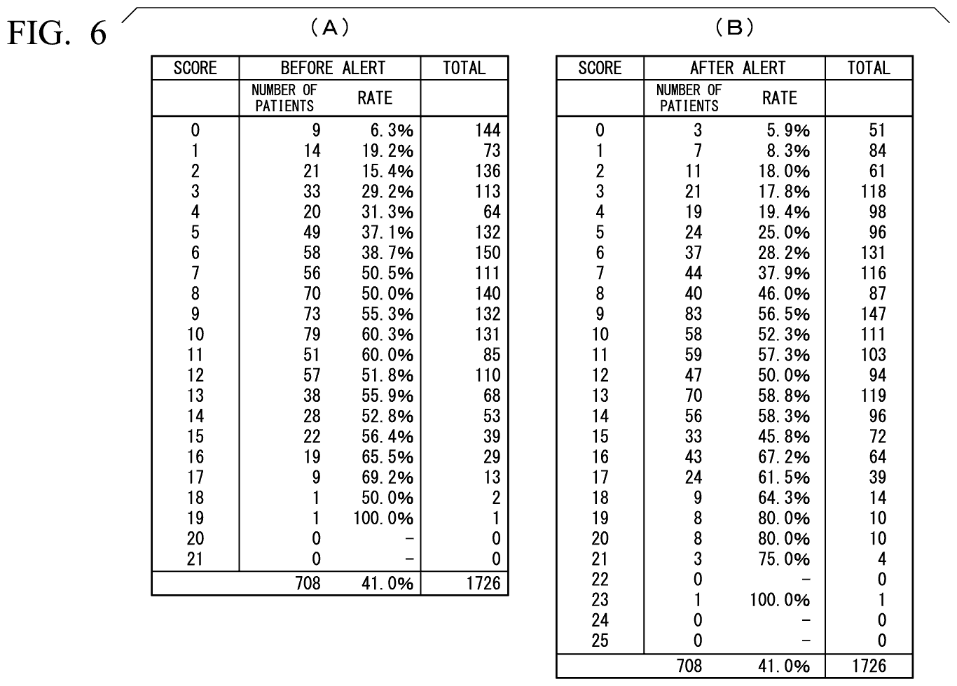

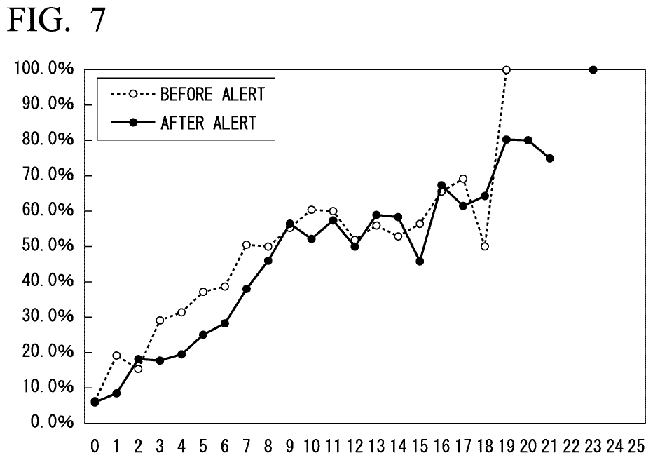

FIG. 6 is a diagram showing a relationship between the severity evaluation score, the number of patients, a severe case rate of a disease, and the number of cases in the example of the monitoring system 1 according to the second embodiment. Specifically, (A) of FIG. 6 shows a relationship between a score-before-alert and the number of patients or the like in the example of the monitoring system 1 according to the second embodiment, and (B) of FIG. 6 shows a relationship between a score-after-alert and the number of patients or the like in the example of the monitoring system 1 according to the second embodiment. FIG. 7 is a diagram comparing a relationship between the severity evaluation score (horizontal axis) and the severe case rate of the disease (vertical axis) in the example of the monitoring system 1 according to a second embodiment with the score-before-alert and the score-after-alert.

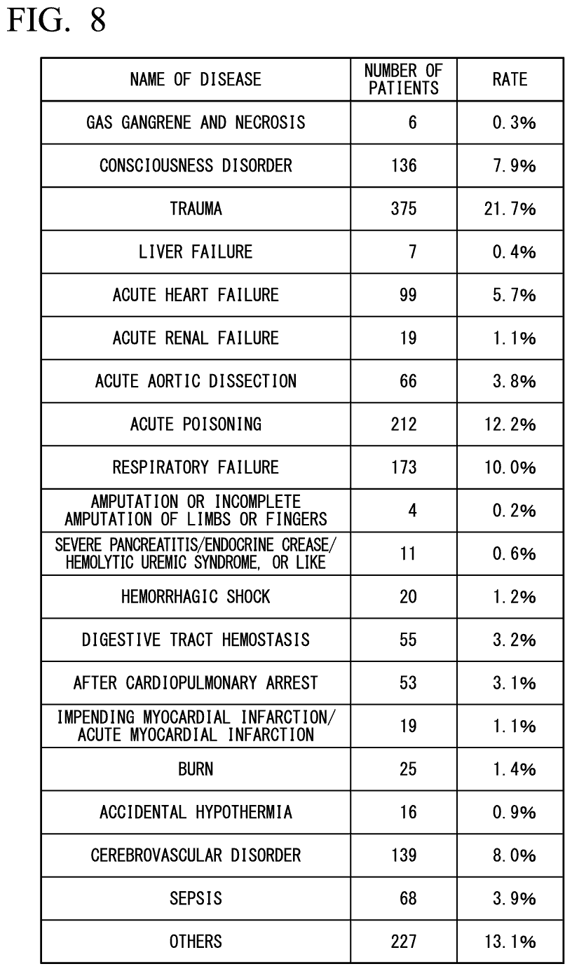

FIG. 8 is a diagram showing a disease to which the monitoring system 1 of the second embodiment is applied for monitoring a severity in the example of the second embodiment of the monitoring system 1.

As shown in FIG. 8, the disease to which the monitoring system 1 according to the second embodiment can be applied for monitoring the severity includes gas gangrene and necrosis, consciousness disorder, trauma, liver failure, acute heart failure, acute renal failure, acute aortic dissection, acute poisoning, respiratory failure, amputation or incomplete amputation of limbs or fingers, severe pancreatitis/endocrine crease/hemolytic uremic syndrome, or the like, hemorrhagic shock, digestive tract bleeding, after cardiopulmonary arrest, impending myocardial infarction/acute myocardial infarction, burn, accidental hypothermia, cerebrovascular disorder, sepsis, and the like.

Hereinabove, although the embodiments of the present invention are described in detail with reference to the drawings, a specific configuration is not limited to the embodiments and can be appropriately modified without departing from the gist of the present invention. The configurations described above in each of the embodiments and each of the examples may be combined.

All or a part of the monitoring system 1 according to the embodiment described above may be realized by dedicated hardware, or may be realized by a memory and a microprocessor.

All or the part of the monitoring system 1 may include a memory and a central processing unit (CPU), and may realize a function thereof in such a manner that a program for realizing a function of each unit included in each system is loaded to and executed by the memory.

A program for realizing all or a part of the functions of the monitoring system 1 may be recorded on a computer-readable recording medium, and the program recorded on the recording medium may be read and executed by a computer system to carry out processing of each unit. The “computer system” herein includes an OS and hardware such as a peripheral device. In addition, the “computer system” includes a homepage providing environment (or a display environment) in a case where a WWW system is used.

In addition, the “computer-readable recording medium” refers to a portable medium such as a flexible disk, a magneto-optical disk, a ROM, and a CD-ROM, and a storage device such as a hard disk built in the computer system. Furthermore, the “computer-readable recording medium” includes a medium which dynamically holds the program for a short period of time as in a communication line in a case where the program is transmitted through a network such as the Internet or a communication line such as a telephone line, and a medium which holds the program for a certain period of time as in a volatile memory inside the computer system serving as a server or a client in that case. In addition, the program described above may be provided to realize a part of the functions described above, or may be realized by combining the functions described above with the program previously recorded in the computer system.

### REFERENCE SIGNS LIST

1: Monitoring system
1A: Wearable terminal
1A1: Input unit
1A2: Detection unit
1A21: Superficial body temperature detection unit
1A22: Heart rate detection unit
1A23: Blood pressure detection unit
1A24: Blood oxygen concentration saturation detection unit
1A25: Reaction detection unit
1A3: Score calculation unit
1A31: Score-before-alert calculation unit
1A32: Score-after-alert calculation unit
1A4: Alert output unit
1A5: Transmission unit
1B: Management server
1B1: Reception unit
1B2: Output unit

1. A monitoring system that includes a wearable terminal and a management server to monitor a wearer of the wearable terminal,
wherein the wearable terminal includes:
a detection unit configured to detect biological information of the wearer of the wearable terminal;
a score calculation unit configured to calculate a severity evaluation score used for evaluating a severity of the wearer of the wearable terminal, based on the biological information of the wearer of the wearable terminal detected by the detection unit;
an alert output unit configured to output a consciousness disorder evaluation alert used for evaluating a consciousness disorder of the wearer of the wearable terminal; and
a transmission unit configured to transmit the severity evaluation score calculated by the score calculation unit, and the biological information of the wearer of the wearable terminal used for calculating the severity evaluation score, to the management server,
the management server includes:
a reception unit configured to receive the severity evaluation score, and the biological information of the wearer of the wearable terminal used for calculating the severity evaluation score, which are transmitted by the transmission unit; and
an output unit configured to output the severity evaluation score, and the biological information of the wearer of the wearable terminal used for calculating the severity evaluation score, which are received by the reception unit, and
in a case where the severity evaluation score, which does not include the evaluation of the consciousness disorder, calculated by the score calculation unit is equal to or more than a threshold value,
the alert output unit outputs the consciousness disorder evaluation alert,
the detection unit detects the biological information of the wearer of the wearable terminal and a reaction of the wearer of the wearable terminal to the consciousness disorder evaluation alert output by the alert output unit,
the score calculation unit calculates the severity evaluation score which includes the evaluation of the consciousness disorder, based on the biological information of the wearer of the wearable terminal and the reaction of the wearer of the wearable terminal to the consciousness disorder evaluation alert output by the alert output unit, which are detected by the detection unit, and
the transmission unit transmits the severity evaluation score, which includes the evaluation of the consciousness disorder, calculated by the score calculation unit, and the biological information of the wearer of the wearable terminal and the reaction of the wearer of the wearable terminal to the consciousness disorder evaluation alert used for calculating the severity evaluation score which includes the evaluation of the consciousness disorder, to the management server.

2. The monitoring system according to claim 1,
wherein the biological information of the wearer of the wearable terminal detected by the detection unit includes a superficial body temperature of the wearer of the wearable terminal, a heart rate of the wearer of the wearable terminal, a mean blood pressure of the wearer of the wearable terminal, a blood oxygen concentration saturation of the wearer of the wearable terminal, and the reaction of the wearer of the wearable terminal to the consciousness disorder evaluation alert output by the alert output unit.

3. The monitoring system according to claim 2,
wherein the wearable terminal includes
an input unit configured to accept an input operation of an age of the wearer of the wearable terminal,
the detection unit includes
a superficial body temperature detection unit configured to detect the superficial body temperature of the wearer of the wearable terminal,
a heart rate detection unit configured to detect the heart rate of the wearer of the wearable terminal,
a blood pressure detection unit configured to detect the mean blood pressure of the wearer of the wearable terminal,
a blood oxygen concentration saturation detection unit configured to detect the blood oxygen concentration saturation of the wearer of the wearable terminal, and
a reaction detection unit configured to detect the reaction of the wearer of the wearable terminal to the consciousness disorder evaluation alert output by the alert output unit, and
the score calculation unit
calculates the severity evaluation score which does not include the evaluation of the consciousness disorder, based on the age of the wearer of the wearable terminal accepted by the input unit, the superficial body temperature of the wearer of the wearable terminal detected by the superficial body temperature detection unit, the heart rate of the wearer of the wearable terminal detected by the heart rate detection unit, the mean blood pressure of the wearer of the wearable terminal detected by the blood pressure detection unit, and the blood oxygen concentration saturation of the wearer of the wearable terminal detected by the blood oxygen concentration saturation detection unit, and
calculates the severity evaluation score which includes the evaluation of the consciousness disorder, based on the age of the wearer of the wearable terminal accepted by the input unit, the superficial body temperature of the wearer of the wearable terminal detected by the superficial body temperature detection unit, the heart rate of the wearer of the wearable terminal detected by the heart rate detection unit, the mean blood pressure of the wearer of the wearable terminal detected by the blood pressure detection unit, the blood oxygen concentration saturation of the wearer of the wearable terminal detected by the blood oxygen concentration saturation detection unit, and the reaction of the wearer of the wearable terminal to the consciousness disorder evaluation alert detected by the reaction detection unit.

4. The monitoring system according to claim 3,
wherein the score calculation unit
calculates any of 0 points, 1 point, 3 points, 5 points, and 6 points as an age score, based on the age of the wearer of the wearable terminal accepted by the input unit,
calculates any of 0 points, 1 point, 2 points, 3 points, and 4 points as a superficial body temperature score, based on the superficial body temperature of the wearer of the wearable terminal detected by the superficial body temperature detection unit,
calculates any of 0 points, 2 points, 3 points, and 4 points as a heart rate score, based on the heart rate of the wearer of the wearable terminal detected by the heart rate detection unit,
calculates any of 0 points, 2 points, 3 points, and 4 points as a mean blood pressure score, based on the mean blood pressure of the wearer of the wearable terminal detected by the blood pressure detection unit,
calculates any of 0 points, 1 point, 3 points, and 4 points as a blood oxygen concentration saturation score, based on the blood oxygen concentration saturation of the wearer of the wearable terminal detected by the blood oxygen concentration saturation detection unit,
calculates any of 0 points, 1 point, 3 points, and 4 points as a consciousness disorder score, based on the reaction of the wearer of the wearable terminal to the consciousness disorder evaluation alert detected by the reaction detection unit, and
calculates the severity evaluation score which does not include the evaluation of the consciousness disorder by adding the age score, the superficial body temperature score, the heart rate score, the mean blood pressure score, and the blood oxygen concentration saturation score, and calculates the severity evaluation score which includes the evaluation of the consciousness disorder by adding the age score, the superficial body temperature score, the heart rate score, the mean blood pressure score, the blood oxygen concentration saturation score, and the consciousness disorder score.

5. The monitoring system according to claim 4,
wherein the score calculation unit
calculates 0 points as the age score in a case where the age of the wearer of the wearable terminal is less than 45 years,
calculates 1 point as the age score in a case where the age of the wearer of the wearable terminal is 45 years or more and 54 years or less,
calculates 3 points as the age score in a case where the age of the wearer of the wearable terminal is 55 years or more and 64 years or less,
calculates 5 points as the age score in a case where the age of the wearer of the wearable terminal is 65 years or more and 74 years or less, and
calculates 6 points as the age score in a case where the age of the wearer of the wearable terminal is 75 years or more.

6. The monitoring system according to claim 5,
wherein the score calculation unit
calculates 0 points as the superficial body temperature score in a case where the superficial body temperature of the wearer of the wearable terminal is 36.0° C. or higher and 38.4° C. or lower,
calculates 1 point as the superficial body temperature score in a case where the superficial body temperature of the wearer of the wearable terminal is 38.5° C. or higher and 38.9° C. or lower, or is 34.0° C. or higher and 35.9° C. or lower,
calculates 2 points as the superficial body temperature score in a case where the superficial body temperature of the wearer of the wearable terminal is 32.0° C. or higher and 33.9° C. or lower,
calculates 3 points as the superficial body temperature score in a case where the superficial body temperature of the wearer of the wearable terminal is 39.0° C. or higher and 40.9° C. or lower, or is 30.0° C. or higher and 31.9° C. or lower, and
calculates 4 points as the superficial body temperature score in a case where the superficial body temperature of the wearer of the wearable terminal is 41.0° C. or higher or lower than 30.0° C.

7. The monitoring system according to claim 6,
wherein the score calculation unit
calculates 0 points as the heart rate score in a case where the heart rate (beats/min) of the wearer of the wearable terminal is 70 or more and 109 or less,
calculates 2 points as the heart rate score in a case where the heart rate (beats/min) of the wearer of the wearable terminal is 110 or more and 139 or less, or is 55 or more and 69 or less,
calculates 3 points as the heart rate score in a case where the heart rate (beats/min) of the wearer of the wearable terminal is 140 or more and 179 or less, or is 40 or more and 54 or less, and
calculates 4 points as the heart rate score in a case where the heart rate (beats/min) of the wearer of the wearable terminal is 180 or more or less than 40.

8. The monitoring system according to claim 7,
wherein the score calculation unit
calculates 0 points as the mean blood pressure score in a case where the mean blood pressure (mmHg) of the wearer of the wearable terminal is 70 or more and 109 or less,
calculates 2 points as the mean blood pressure score in a case where the mean blood pressure (mmHg) of the wearer of the wearable terminal is 110 or more and 129 or less, or is 50 or more and 69 or less,
calculates 3 points as the mean blood pressure score in a case where the mean blood pressure (mmHg) of the wearer of the wearable terminal is 130 or more and 159 or less, and
calculates 4 points as the mean blood pressure score in a case where the mean blood pressure (mmHg) of the wearer of the wearable terminal is 160 or more or less than 50.

9. The monitoring system according to claim 8,
wherein the score calculation unit
calculates 0 points as the blood oxygen concentration saturation score in a case where the blood oxygen concentration saturation (%) of the wearer of the wearable terminal is 94 or more,
calculates 1 point as the blood oxygen concentration saturation score in a case where the blood oxygen concentration saturation (%) of the wearer of the wearable terminal is 91 or more and 93 or less,
calculates 3 points as the blood oxygen concentration saturation score in a case where the blood oxygen concentration saturation (%) of the wearer of the wearable terminal is 88 or more and 90 or less, and
calculates 4 points as the blood oxygen concentration saturation score in a case where the blood oxygen concentration saturation (%) of the wearer of the wearable terminal is less than 88.

10. The monitoring system according to claim 9,
wherein the score calculation unit
calculates 0 points as the consciousness disorder score in a case where the reaction of the wearer of the wearable terminal to the consciousness disorder evaluation alert corresponds to “0: clear consciousness” of Japan Coma Scale (JCS),
calculates 1 point as the consciousness disorder score in a case where the reaction of the wearer of the wearable terminal to the consciousness disorder evaluation alert corresponds to “1: orientation is maintained but consciousness is not clear”, “2: disorientation”, or “3: unable to say own name and date of birth” of JCS,
calculates 3 points as the consciousness disorder score in a case where the reaction of the wearer of the wearable terminal to the consciousness disorder evaluation alert corresponds to “10: opening eyes in response to normal call”, “20: opening eyes in response to loud call, strong shake, or the like”, or “30: opening eyes with difficulty in response to continued call in a presence of pain stimulation” of JCS, and
calculates 4 points as the consciousness disorder score in a case where the reaction of the wearer of the wearable terminal to the consciousness disorder evaluation alert corresponds to “100: acting such as pushing away in response to pain”, “200: moving hands and feet or frown in response to pain stimulation”, or “300: no response at all to pain stimulation” of JCS.

11. The monitoring system according to claim 10,
wherein in a case where the severity evaluation score which does not include the evaluation of the consciousness disorder obtained by adding the age score, the superficial body temperature score, the heart rate score, the mean blood pressure score, and the blood oxygen concentration saturation score is 3 points or more, the alert output unit outputs the consciousness disorder evaluation alert.

12. The monitoring system according to claim 10,
wherein in a case where the severity evaluation score which does not include the evaluation of the consciousness disorder obtained by adding the age score, the superficial body temperature score, the heart rate score, the mean blood pressure score, and the blood oxygen concentration saturation score is 5 points or more, the alert output unit outputs the consciousness disorder evaluation alert.

13. The monitoring system according to claim 11,
wherein the monitoring system is used for preventing occurrence of heat stroke or for preventing aggravation of heat stroke of the wearer of the wearable terminal.

14. The monitoring system according to claim 11,
wherein the monitoring system is used for preventing aggravation of a disease of the wearer of the wearable terminal.

15. A wearable terminal used for monitoring a wearer of the wearable terminal, the wearable terminal comprising:
a detection unit configured to detect biological information of the wearer of the wearable terminal;
a score calculation unit configured to calculate a severity evaluation score used for evaluating a severity of the wearer of the wearable terminal, based on the biological information of the wearer of the wearable terminal detected by the detection unit;
an alert output unit configured to output a consciousness disorder evaluation alert used for evaluating a consciousness disorder of the wearer of the wearable terminal; and
a transmission unit configured to transmit the severity evaluation score calculated by the score calculation unit, and the biological information of the wearer of the wearable terminal used for calculating the severity evaluation score, to a management server,
wherein in a case where the severity evaluation score, which does not include the evaluation of the consciousness disorder, calculated by the score calculation unit is equal to or more than a threshold value,
the alert output unit outputs the consciousness disorder evaluation alert,
the detection unit detects the biological information of the wearer of the wearable terminal and a reaction of the wearer of the wearable terminal to the consciousness disorder evaluation alert output by the alert output unit,
the score calculation unit calculates the severity evaluation score which includes the evaluation of the consciousness disorder, based on the biological information of the wearer of the wearable terminal and the reaction of the wearer of the wearable terminal to the consciousness disorder evaluation alert output by the alert output unit, which are detected by the detection unit,
the transmission unit transmits the severity evaluation score, which includes the evaluation of the consciousness disorder, calculated by the score calculation unit, and the biological information of the wearer of the wearable terminal and the reaction of the wearer of the wearable terminal to the consciousness disorder evaluation alert used for calculating the severity evaluation score which includes the evaluation of the consciousness disorder, to the management server,
a reception unit of the management server receives the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are transmitted by the transmission unit, and
an output unit of the management server outputs the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are received by the reception unit.

16. A monitoring method of monitoring a wearer of a wearable terminal, the monitoring method comprising:
a detection step of detecting biological information of the wearer of the wearable terminal, via the wearable terminal;
a score calculation step of calculating a severity evaluation score used for evaluating a severity of the wearer of the wearable terminal, based on the biological information of the wearer of the wearable terminal detected in the detection step, by the wearable terminal;
an alert output step of outputting a consciousness disorder evaluation alert used for evaluating a consciousness disorder of the wearer of the wearable terminal, by the wearable terminal;
a transmission step of transmitting the severity evaluation score calculated in the score calculation step, and the biological information of the wearer of the wearable terminal used for calculating the severity evaluation score, to a management server, by the wearable terminal;
a reception step of receiving the severity evaluation score, and the biological information of the wearer of the wearable terminal used for calculating the severity evaluation score, which are transmitted in the transmission step, by the management server; and
an output step of outputting the severity evaluation score, and the biological information of the wearer of the wearable terminal used for calculating the severity evaluation score, which are received in the reception step, by the management server,
wherein in a case where the severity evaluation score, which does not include the evaluation of the consciousness disorder, calculated in the score calculation step is equal to or more than a threshold value,
in the alert output step, the consciousness disorder evaluation alert is output,
in the detection step, the biological information of the wearer of the wearable terminal and a reaction of the wearer of the wearable terminal to the consciousness disorder evaluation alert output in the alert output step are detected,
in the score calculation step, the severity evaluation score which includes the evaluation of the consciousness disorder is calculated, based on the biological information of the wearer of the wearable terminal and the reaction of the wearer of the wearable terminal to the consciousness disorder evaluation alert output in the alert output step, which are detected in the detection step, and
in the transmission step, the severity evaluation score, which includes the evaluation of the consciousness disorder, calculated in the score calculation step, and the biological information of the wearer of the wearable terminal and the reaction of the wearer of the wearable terminal to the consciousness disorder evaluation alert used for calculating the severity evaluation score which includes the evaluation of the consciousness disorder are transmitted to the management server.

17. A program causing a computer mounted in a wearable terminal used for monitoring a wearer of the wearable terminal to execute:
a detection step of detecting biological information of the wearer of the wearable terminal;
a score calculation step of calculating a severity evaluation score used for evaluating a severity of the wearer of the wearable terminal, based on the biological information of the wearer of the wearable terminal detected in the detection step;
an alert output step of outputting a consciousness disorder evaluation alert used for evaluating a consciousness disorder of the wearer of the wearable terminal; and
a transmission step of transmitting the severity evaluation score calculated in the score calculation step, and the biological information of the wearer of the wearable terminal used for calculating the severity evaluation score, to a management server,
wherein in a case where the severity evaluation score, which does not include the evaluation of the consciousness disorder, calculated in the score calculation step is equal to or more than a threshold value,
in the alert output step, the consciousness disorder evaluation alert is output,
in the detection step, the biological information of the wearer of the wearable terminal and a reaction of the wearer of the wearable terminal to the consciousness disorder evaluation alert output in the alert output step are detected,
in the score calculation step, the severity evaluation score which includes the evaluation of the consciousness disorder is calculated, based on the biological information of the wearer of the wearable terminal and the reaction of the wearer of the wearable terminal to the consciousness disorder evaluation alert output in the alert output step, which are detected in the detection step,
in the transmission step, the severity evaluation score, which includes the evaluation of the consciousness disorder, calculated in the score calculation step, and the biological information of the wearer of the wearable terminal and the reaction of the wearer of the wearable terminal to the consciousness disorder evaluation alert used for calculating the severity evaluation score which includes the evaluation of the consciousness disorder are transmitted to the management server,
a reception unit of the management server receives the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are transmitted in the transmission step, and
an output unit of the management server outputs the severity evaluation score which includes the evaluation of the consciousness disorder, and the biological information of the wearer of the wearable terminal and the reaction of the wearer of the wearable terminal to the consciousness disorder evaluation alert used for calculating the severity evaluation score which includes the evaluation of the consciousness disorder, which are received by the reception unit.
